(19) United States
(12) Patent Application Publication
(10) Pub. No.: US 2007/0196277 A1
(43) Pub. Date: Aug. 23, 2007

(54) Compositions and Methods for the Direct Therapy of Tumors

(76) Inventors: Victor A. Levin, Houston, TX (US); Ehud Mendel, Columbus, OH (US)

Correspondence Address: FULBRIGHT & JAWORSKI L.L.P., 600 CONGRESS AVE., SUITE 2400, AUSTIN, TX, 78701, US

(21) Appl. No.: 11/625,520

(22) Filed: Jan. 22, 2007

Related U.S. Application Data
(60) Provisional application No. 60/760,980, filed on Jan. 20, 2006.

(51) Int. Cl. A61K 51/04

(52) U.S. Cl. 424/1.65

(57) ABSTRACT

The present invention provides methods for the treatment of tumors, such as spinal metastases, using compositions that permit the introduction of chemotherapeutics intratumorally while concurrently visualizing the procedure. Thus, in certain embodiments, the invention concerns a composition comprising a chemotherapeutic, an alcohol and an iodinated contrast agent (e.g., iodouracil) that may be introduced into the tumor by direct injection through the aid of an imaging device, such as a CT scanner, x-ray machine, fluoroscope or the like. The ability to visualize drug solution permits the faithful introduction of the drug directly into the tumor, as opposed to surrounding tissues, which is enabled through the use of the contrast medium.

### BACKGROUND OF THE INVENTION

The present application claims the benefit of provisional application U.S. Ser. No. 60/760,980, filed Jan. 20, 2006, the specification of which is incorporated herein by reference.

1. Field of the Invention

The present invention relates generally to the fields of medicine and cancer therapy. More particularly, it concerns the treatment of spinal metastases by the administration of BCNU-ethanol.

2. Description of Related Art

Spinal tumors are classified by anatomical location. Extradural metastases account for some 95% of secondary spinal tumors. These lesions arise through blood-born spread of cancerous cells or by direct extension of the primary tumor. Most extradural tumors are metastases to the vertebral bodies, but some lymphomas and tumors from Hodgkin's disease may be in the epidural space without bone involvement. The majority of the metastases are multiple and eventually encroach upon the epidural space. Metastatic spinal tumors seldom breach the dura, but intradural and intramedullary lesions can invade dura. The primary sources of metastatic neoplasms to the spinal axis vary among the published series with 65% coming from carcinoma of the breast, lung, and prostate (Perrin et al., 2002). Renal cell carcinoma and myeloma account for another 10% of spinal metastases (Perrin et al., 2002).

The majority of patients with systemic cancer develop skeletal metastases with the spine being the most commonly involved bone group. Spinal metastases are present in 40% of patients who die of cancer. Symptomatic spinal metastases occur in approximately 5% to 10% of cancer patients sometime in the course of their disease (Bilsky and Vitaz, 2005). These symptomatic spinal metastases most often involve the thoracic spine (in 70% of cases) followed by the lumbar (20%) and cervical segments (10%) (Perrin et al., 2002). In the United States each year it would be expected, therefore, that over 50,000 patients would be expected to have symptomatic vertebral metastases (Gokaslan et al., 1998).

These metastases can lead to debilitating pain and weakness that cause a marked deterioration in quality of life. Pain, involving the back or neck is common, can be biologic due to tumor growth and invasion or mechanical due to structural instability of vertebrae and vertebral facets (Perrin et al., 2002; Bilsky and Vitaz, 2005). Other symptoms such as localized sensory loss or sphincter disturbances can result from compressive neuropathy or myelopathy (Perrin et al., 2002; Bilsky and Vitaz, 2005). The diagnosis of vertebral metastasis can be made by MRI or CT, with MRI being the more sensitive and specific imaging technique (Bilsky and Vitaz, 2005; Schiff et al., 1998; Moulopoulos et al., 1996; Zhou et al., 2002)). Because pain and functional neurologic status are usually involved in spinal metastases, pain scales and neurological function scoring systems are usually used in assessing these patients and evaluating treatment modalities (Bilsky and Vitaz, 2005).

The treatment for vertebral metastases is primarily focal radiation therapy and secondarily surgery for spinal stabilization and relief of pain, when possible (Schiff et al., 1995; Gilbert et al., 1978; Maranzano and Latini, 1995; Maranzano et al., 1991). The place of systemic chemotherapy is dependent on the histology of the spinal metastasis (Bilsky and Vitaz, 2005). There is no literature on regional or intratumoral chemotherapy for vertebral metastases.

One approach that has been suggested for the treatment of tumor and metastatic tumors is the use of a chemotherapeutic agent, particularly BCNU, dissolved in absolute ethanol (see, e.g., U.S. Pat. Nos. 5,051,257; 5,162,115 and 6,753,005). Indeed, such product, known as DTI-015, is currently in clinical trials for the treatment of brain cancer and liver cancer, in which it is administered by intratumoral injection using computed tomographic (CT) guiding. While initial results suggest that the DTI-015 formulation has promise and is efficacious, its primary drawback is the inability to visualize the solution as it is being injected into the tumor and thereafter. Thus, it is difficult if not impossible to properly target the tumor and faithfully inject the drug into the tumor (as opposed to surrounding tissues) and thereafter determine the degree of intratumoral dissemination of the solution (and, hence, drug) and difficult to administer the solution such that the tumor body is properly and completed infused with the solution. While U.S. Pat. No. 6,753,005 mentions the possibility of including in the treatment solutions iodinated contrast agents that are CT opaque, it mentions only high molecular weight (typically greater than 650 daltons) iodinated compounds that are highly iodinated (tri-iodinated and higher). Unfortunately, such compounds have been found by the present inventor to not be useful in actual practice, in the due to their high molecular weight and significant iodination, such compounds do not freely move with the ethanol solvent front and thus do not provide a suitable means for visualizing the ethanol and drug distribution.

### SUMMARY OF THE INVENTION

Accordingly, the present invention is directed generally to diagnostic and therapeutic formulations that permit physicians to directly introduce desired therapeutic molecules directly into neoplastic tissues, such as tumors and particularly metastatic tumors, while simultaneously visualizing both the introduction of the agent (i.e., by visualizing the injection means/syringe), as well as visualizing the solvent front and, hence, agent, as it disseminates through the tumor. The advantages of such an approach lies in the ready visualization during administration (e.g., by CT scan) as well as ready identification and observation of the treated tumor during subsequent follow-up therapies or patient evaluation.

Thus, one aspect of the invention concerns a pharmaceutical composition, that may also be referred to as a therapeutic or diagnostic (i.e., imaging) solution, which includes a C1-C4 alcohol having a partition coefficient of at least 0.1 and a mono- or di-iodinated contrast agent dissolved therein, wherein the contrast agent is soluble in alcohol, DMSO or alcohol/DMSO and has a molecular weight of less than 500. Exemplary alcohols include methanol, ethanol, 1-propanol, 2-propanol, 1-propene-3-ol, t-butanol, and the like. Of course, due to potential toxicity concerns certain alcohols (e.g., methanol) will not be preferred, whereas ethanol, and particularly absolute ethanol, will be the most preferred. When the foregoing solutions are used for therapeutic applications of tumors the will, of course, include a selected agent such as a chemotherapeutic agent.

It have been discovered by the inventors that an important aspect of the invention is the selection of a contrast agent that has the desirable property of remaining entrained in the alcohol solution, e.g., capable of moving with the alcohol solvent front, as it is being introduced into the tumor and disseminated therein, and which contrast agent is capable of being readily detected using conventional imaging techniques, such as CT guiding lodinated contrast agents are well studied and most are generally pharmacologically acceptable, and such agents fit the criteria of being readily detected by, for example, CT. However, the inventors have discovered that a discrete subclass of iodide-containing compound or known contrast agents are preferred in that certain such compounds are not capable of moving with the solvent front. For this reason, iodide-containing compounds (“contrast agents”) having a molecular weight of less that about 500, and more preferably less than about 450 and still more preferably less than about 400, are particularly preferred for practice in connection with the present invention.

Furthermore, in that it is highly desirable that the contrast agent remain entrained in the alcohol front, it is preferable that the contrast agent be generally soluble in alcohol (preferably ethanol), DMSO (or a similar solubilizing agent) or DMSO/alcohol mixtures. Other considerations for selecting preferred iodide contrast agents will be to employ those that are either mono- or di-iodinated (as compared to the more typical iodide contrast agents that are multi-iodinated). Due to their molecular weight, iodide moieties add substantially to the molecular weight of the contrast agent and, perhaps separate from their molecular weight, tend to reduce the ability of the contrast agent to travel with the alcohol solvent front. Hence, mono-iodinated contrast agents will generally be more preferred than di-iodinated agents, and so on.

A still further consideration in the selection of preferred contrast agents is the degree of charge carried by the molecule at physiologic pH, with the lower charge and, preferably no charge, at physiologic pH being particularly preferred.

Exemplary iodinated contrast agents believed to be useful in the practice of the invention include mono- and di-iodinated imides, purines and pyrimidines; benzyls, phenols or benzoic acids; N-pyridones and glycerols, with mono- and di-iodinated succinamide, iodinated glycerol, iodouracil or iofetamine being preferred, with iodouracil being particularly preferred.

The antineoplastic agents that can be formulated into effective direct delivery antineoplastic solutions according to the present invention include chemotherapeutics, biotherapeutics and radiotherapeutics exhibiting cytotoxic or cytostatic activity. By using the methods disclosed, therapeutically effective doses of substantially any antineoplastic agent solute can be delivered throughout an at least substantial volume of the mass of a tumor without regard to the exact nature of the antineoplastic agent or its molecular mechanism of action (excluding the case, of course, wherein extratumoral activation of the agent is necessary). Preferred antineoplastic agents are those that are non-ionized, low molecular weight (e.g., less that 500 daltons) and sufficiently lipophilic to be soluble in alcohol or DMSO/alcohol. Useful chemotherapeutic agents include alkylating agents, such as platinum coordination compounds, nitrogen mustards, nitrosoureas, ethyleneimine derivatives, alkyl sulfonates and triazenes; antimetabolites, such as folic acid analogs, pyrimidine analogs, and purine analogs; vinca alkaloids; antibiotics; hormones, such as adrenocorticosteroids, progestins, estrogens and androgens; and miscellaneous subclasses, such as methyl hydrazine derivatives and amidoximes. Useful biotherapeutics include monoclonal antibodies; monoclonal antibody cytotoxic conjugates of drugs and toxins, for example, ricin A chain or pokeweed antiviral protein; cytokines; biologic response modifiers; lymphokines; interferons; interleukins; growth factors; growth factor inhibitors; natural, recombinant, and synthetic proteins, enzymes, peptides, and nucleic acids and their functional equivalents. Useful radiotherapeutics include radionuclides of iodine or bromine, radioisotope labeled monoclonal antibodies, other radioisotope labeled tumor homing agents and metabolites exhibiting specific preference for tumors, and radioisotope labeled agents not displaying any tumor localization preference but having the solubility characteristics of the present invention.

The nitrosoureas, such as BCNU (1,3-bis(2-chloroethyl)-1-nitrosourea), are particularly preferred for use in the practice of the present invention. Other names for BCNU include: carmustine, N,N′-bis(2-chloroethyl)-N-nitrosourea, NSC-409962 and WR-139021. BCNU is described, for example, in Hammoud et al. (2003), Koositra et al. (1989), Kitamura et al. (1996), U.S. Pat. Nos. 6,753,005, 5,162,115, and 5,051,257. BCNU is highly soluble in ethanol and lipids, and poorly soluble in water [Physicians Desk Reference 1995]. It is also stable and bioavailable in the absolute ethanol direct delivery vehicle (U.S. Pat. Nos. 5,162,115 and 5,051,257; Chan and Zackheim, 1973; Laskar and Ayres, 1977; Levin and Levin, 1989). When the ethanol vehicle penetrates the tumor it transports high levels of active BCNU rapidly throughout the tumor. BCNU may be obtained as BiCNU from Bristol-Myers Squibb Oncology, P.O. Box 4500, Princeton, N.J. 08543-4500 as BCNU powder in one vial. The ethanol can be obtained as Dehydrated Alcohol for injection, U.S.P.

Depending on the particular contrast agent that is employed (and its degree of solubility in alcohol at desirable concentrations), one may find it useful to further include a solubilizing agent. Particularly preferred are generally non-toxic solubilizing agents such as DMSO, a pharmacologically acceptable dipolar aprotic solvent. Other such solvents include dimethylformamide, dimethylacetamide, N-methyl-2-pyrrolidone, HMPA, and the like, although such solvents are much less preferred due to their potential toxicity.

Generally, it is proposed that desirable concentrations of the chemotherapeutic agent (depending on its effective dose, solubility, etc.) will be on the order of 5 mg/ml to 60 mg/ml (w/v), with 10 mg/ml to 50 mg/ml (w/v) being more preferred, and 20 mg/ml to 40 mg/ml (w/v), and particularly 30 mg/ml, being most preferred.

Similarly, while not believed to be critical, the iodinated contrast agent will typically comprises about 1% to 10% (w/v) of the composition, with about 2% to 8% being even more preferred.

The compositions of the invention are preferably formulated in a sterile manner and placed into a sterile vessel in a metered amount. Such vessels may include a sterile vial or ampoule, preferably with a security seal and/or septum.

The present invention is, in further embodiments, directed to a method of treating or visualizing a tumor in a subject using the compositions of the present invention. While the subject will typically be a human, the present invention is also applicable to the treatment or visualization of animal tumors. Generally speaking, visualization and/or treatment of the tumor is effected by directly injecting the composition into the tumor, or into spaces adjacent or near the tumor, while simultaneously visually the process by means of an appropriate visualization means (e.g., CT, fluorography, X-ray, MRI, etc.) Furthermore, while the invention is not limited in terms of the amount or volume of the composition that is introduced into the tumor, the inventors believe that particular advantages will be realized by calculating and administering an amount of solution that corresponds to as low as about 25% but usually about 50% to about 100% of the volume of the individual tumor.

The applicability of the present invention is not limited to any particular tumor or tumor type. The tumor may be a precancerous tumor, a benign tumor (e.g., a uterine chorioangioma) or a cancerous tumor (e.g., a metastatic tumor). Part or all of the tumor may be non-resectable and/or difficult to resect. The invention includes, for example, the visualization or treatment of tumors present in liver, a lymph node, urinary tract, lung, central nervous system (brain and spinal cord) and dural covering, head and neck, urogenital system, uterus, vertebra, soft tissue, skin, cartilage, bone, or gastrointestinal tract. Nonetheless, it is contemplated that the invention will find particular applicability to the treatment or visualization of metastatic tumors, such as extradural metastasis, an intradural metastasis, an intramedullary metastasis, and an intravertebral metastasis. Still more preferred will the those metastatic tumors that originate from a cancer of the breast, lung, blood forming cells, plasma cells, uterus, prostate, and kidney as well as adenocarcinoma, squamous cell carcinoma, renal cell carcinoma, or a myeloma.

Compositions of the present invention are preferably prepared by solubilizing the iodinated contrast agent with a solubilizing agent such as DMSO or DMSO/alcohol (where needed) and admixing the solubilized iodinated contrast agent with the selected alcohol to form the alcohol contrast agent solution. In such instances, as noted above, the composition will typically be formulated to include from about 1% to about 10% of the iodinated contrast agent per milliliter. Where the formulation is to be used for therapeutic applications as well, the process for preparation will include admixing the alcohol contrast agent composition with a selected chemotherapeutic such as BCNU.

In still further embodiments, it is proposed by the inventors that the present invention will find particular applicability in the therapy of spinal metastatic tumors. Such treatments are not limited to, but may preferably include, the use of a contrast agent in the alcohol/drug mixture. Indeed, it is contemplated that formulations such as DTI-015 (see U.S. Pat. Nos. 5,015,257; 5,162,115 and 6,753,005, all incorporated herein by reference), with or without added contrast agent, can be used advantageously for such purposes. IN such treatment methods, the formulation is preferably directly administered intravertebrally, such as intraspinally to a spinal axis metastasis, to the tumor. While the formulation is preferably injected directly into the tumor, it is believed that some therapeutic benefit will be realized by injection near the tumor. It is contemplated that the tumor may alternatively be an extradural metastasis, an intradural metastasis, or an intramedullary metastasis.

Moreover, it is contemplated that still further benefit to the patient may be realized by subjecting the patient to follow-up cancer therapies subsequent to the direct intratumoral therapy. Exemplary follow-up or secondary therapies may include chemotherapy, focal radiation therapy or a surgery, that will be selected depending on the tumor type, presentation and the like.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The antineoplastic agents that can be formulated into effective direct delivery antineoplastic solutions according to the present invention include chemotherapeutics, biotherapeutics and radiotherapeutics exhibiting cytotoxic or cytostatic activity. By using the methods disclosed, therapeutically effective doses of substantially any antineoplastic agent solute can be delivered throughout an at least substantial volume of the mass of a tumor without regard to the exact nature of the antineoplastic agent or its molecular mechanism of action (excluding the case, of course, wherein extratumoral activation of the agent is necessary). Useful chemotherapeutic agents include alkylating agents, such as platinum coordination compounds, nitrogen mustards, nitrosoureas, ethyleneimine derivatives, alkyl sulfonates and triazenes; antimetabolites, such as folic acid analogs, pyrimidine analogs, and purine analogs; vinca alkaloids; antibiotics; hormones, such as adrenocorticosteroids, progestins, estrogens and androgens; and miscellaneous subclasses, such as methyl hydrazine derivatives and amidoximes. Useful biotherapeutics include monoclonal antibodies; monoclonal antibody cytotoxic conjugates of drugs and toxins, for example, ricin A chain or pokeweed antiviral protein; cytokines; biologic response modifiers; lymphokines; interferons; interleukins; growth factors; growth factor inhibitors; natural, recombinant, and synthetic proteins, enzymes, peptides, and nucleic acids and their functional equivalents. Useful radiotherapeutics include radioactive iodine or bromine, radioisotope labeled monoclonal antibodies, other radioisotope labeled tumor homing agents and metabolites exhibiting specific preference for tumors, and radioisotope labeled agents not displaying any tumor localization preference but having the solubility characteristics of the present invention.

Operationally, where cytotoxic, the antineoplastic agent is preferably dissolved in the organic solvent to a concentration such that when a therapeutically effective volume of the antineoplastic solution of the invention is delivered into a neoplastic mass, there results a dose of the agent solute in said mass of at least two logs greater than its tumoricidal dose 50% (TD50), that is, the dose of agent in said mass that kills 50% of the tumor cells. This ensures that a supralethal concentration of the agent is delivered throughout at least a substantial volume of the tumor mass when the resulting antineoplastic solution is injected directly into the substance of the tumor. As the organic vehicle permeates the tumor mass it transports a therapeutically effective, neoplastically lethal concentration of the cytotoxic agent solute therewith. In this way high levels of the cytotoxic antineoplastic agent can be delivered discretely and with relative safety to the tumor mass. In another sense the water miscible organic vehicle component of the solution can be considered to increase the solubility of the agent within the tumor mass, thereby allowing therapeutically effective toxic levels of the antineoplastic agent to invest the tumor. It must also be appreciated that antineoplastic agents with high solubility in the water miscible organic solvent vehicles of the invention will themselves usually tend to have good cellular diffusivity characteristics and can thus, upon intratumoral administration, diffuse relatively efficiently on their own, perhaps even beyond the diffusion zone of the solvent vehicle component. The stability and bioavailability of the antineoplastic agent in the selected solvent vehicle aid in insuring that high levels of active drug permeate the tumor.

Many of the specific antineoplastic agents in current use have been, in part, chosen because of their high water solubilty, thereby allowing them to be administered according to standard prior art delivery techniques. In accordance with the present invention, however, highly effective antineoplastic agents can be designed whose molecular architecture has been optimized for solubility, stability and/or bioavailability in one or more of the water miscible organic solvent vehicles of the invention. The present invention, therefore, provides a particularly efficacious means of delivering highly lipophilic agents directly to the tumor mass.

In many instances, suitable antineoplastic agents already exist or are easily synthesized. For example, well known chemotherapeutic agents useful in the present invention include aceglatone, BCNU, busulfan, CCNU, chlorambucil, cactinomycin, carzinophilin, chlomaphazine, 6-chloropurine, cis-platinum, dactinomycin, demecolcine, ethylenimine quinone, hadacidin, lomustine, mechlorethamine, melphalan, Me-CCNU, plicamycin, mitotane, mycophenolic acid, nitracrine, nogalamycin, streptonigrin, streptozocin, tegafur, tetramin, testolactone, triaziquinone, 2,2′,2″-trichlorotriethylamine, trichodermin, triethylenephosphoramide, triethylenethiophosphoramide, ubenimex, urethan, vinblastine and vincristine. Antineoplastic peptides, proteins, enzymes and the like, which may not inherently possess proper solubility characteristics in their natural form, can be made more soluble in the organic solvent vehicles of the invention by incorporating suitable amino acid residues or sequences in their molecular architectures or by direct chemical modification. Useful radioisotopes include phosphorus-32, yttrium-90, cobalt-60, gold-198, iridium-192, iodine-130, iodine-121, iodine-132, tantalum-182, copper-67, sulfur-35, and sodium-24.

Under some circumstances the water miscible organic solvent vehicles of the invention may exert direct cytotoxic effects themselves or exert a cooperative toxic effect on tumor masses treated therewith when combined with appropriate antineoplastic agents. Of course, mixtures of mutually compatible water miscible organic solvent vehicles as well as mixtures of compatible antineoplastic agent solutes may also be utilized and are specifically contemplated herein.

Antineoplastic agents previously considered too toxic for use with conventional prior art delivery methods may now be found useful under the present invention. This is so because the side toxicities of the resulting antineoplastic solutions are not as severe as can be encountered when delivered by standard extratumoral delivery methods since so little -agent will reach systemic sites of toxicity. Antineoplastic agents may also be tailored to exhibit high stability in the water miscible organic solvent vehicles but be rather unstable in aqueous solution (for instance, BCNU is stable in ethanol but has a half life in serum of only about 15 minutes). In addition to being greatly diluted out, such antineoplastic agents will also be at least partially inactivated before reaching systemic sites of toxicity should the agents permeate the tumor and diffuse into surrounding healthy tissue. Of course, for a given tumor type an antineoplastic agent should be chosen which has a high toxicity for that tumor. In this way, as in the example below, the tumor can be dosed with cytotoxic levels several orders of magnitude greater than the tumoricidal dose 50%.

The nitrosoureas, such as BCNU (1,3-bis(2-chloroethyl)-1-nitrosourea), are particularly preferred for use in the practice of the present invention. Other names for BCNU include: carmustine, N,N′-bis(2-chloroethyl)-N-nitrosourea, NSC-409962 and WR-139021. BCNU is described, for example, in Hammoud et al. (2003), Koositra et al. (1989), Kitamura et al. (1996), U.S. Pat. Nos. 6,753,005, 5,162,115, and 5,051,257. BCNU is highly soluble in ethanol and lipids, and poorly soluble in water [Physicians Desk Reference 1995]. It is also stable and bioavailable in the absolute ethanol direct delivery vehicle (U.S. Pat. Nos. 5,162,115 and 5,051,257; Chan and Zackheim, 1973; Laskar and Ayres, 1977; Levin and Levin, 1989). When the ethanol vehicle penetrates the tumor it transports high levels of active BCNU rapidly throughout the tumor. BCNU may be obtained as BiCNU from Bristol-Myers Squibb Oncology, P.O. Box 4500, Princeton, N.J. 08543-4500 as BCNU powder in one vial. The ethanol can be obtained as Dehydrated Alcohol for injection, U.S.P.

The present invention provides, in certain embodiments, methods for the treatment of spinal metastases comprising injecting vertebral metastases with a mixture .of 1,3-bis(2-chloroethyl)-1-nitrosourea (BCNU) in ethanol. This mixture has been studied as DTI-015, which has been studied by Direct Therapeutics Incorporated. DTI-015 has been extensively studied as an intratumoral treatment for recurrent glioblastoma and other high-grade gliomas (Hassenbusch et al., 2003). The direct injection of BCNU-ethanol mixture into experimental tumors produces a 100- to 1000-fold increase in tumor BCNU over that attainable by intravenous administration (Hamstra et al., 2005). BCNU exerts part of its clinical activity through DNA alkylation and cross-linking. Bodell and colleagues found that they could measure N7-(2-hydroxyethyl)guanine (N7-HOEtG), one of the DNA alkylation products formed by BCNU, in experimental tumors and glioblastoma tumors at the time of surgery (Bodell et al., 2001; Bodell et al., 2003). As an example, they found levels of N7-HOEtG detected in RIF-1 tumors following intra-tumoral administration of BCNU-ethanol mixture were 164-fold higher than levels of N7-HOEtG in the intra-peritoneal BCNU treated tumor samples (Bodell et al., 2003). Bodell also measured the levels of N7-HOEtG in DNA isolated from tumor samples taken from four patients with GBM tumors following stereotactic intratumoral injection with BCNU-ethanol. The level of N7-HOEtG in these samples ranged from 14.7 to 121.9 mumol N7-HOETG/mol DNA within 1 cm of the site of injection. The levels of N7-HOEtG were 0.2 to 0.3 mumol N7-HOETG/mol DNA at 3.5 to 3.9 cm from the site of injection in two patients. The levels of N7-HOEtG in these tumor samples corresponded to BCNU treatment concentrations of 0.02 to 43.0 mM. These studies demonstrate that stereotactic intratumoral injection of DTI-015 into human GBM tumors produces high concentrations of BCNU up to 2.5 cm from the site of injection in some of the tumors. (Bodell et al. 2007)

In the phase I study of BCNU-ethanol mixture in glioblastoma patients, it was found that the maximum tolerated dose (MTD) was 5 ml of ethanol and 240 mg (Hassenbusch et al., 2003). Based on fill volume estimates published for vertebral bodies, it is highly likely that vertebral tumors will occupy less than 6 cc/vertebra (Table 1). If that in fact is the case, then injecting BCNU-ethanol mixture at volumes between 2 and 6 cc may be feasible and therapeutic. Because of this belief, the inventors propose a phase I/II study to determine an optimal ethanol volume and BCNU amount that will be safe for patients and, at the same time to evaluate whether intratumoral injection of BCNU-ethanol mixture can control vertebral tumor growth and alleviate symptoms such as pain, weakness, sensory loss, and bladder or bowel sphincter dysfunction.

TABLE 1
Human vertebral body fill volume estimation based on ⅔ volume
of the anterior vertebral body less 20% volume occupied by the cancellous
bone and less 50% residual marrow. The calculations assume that 50%
of the marrow will remain after injection.

| | L5 | L3 | L1 | T11 | T9 | T7 |
|---|---|---|---|---|---|---|
| Vertebral | 44.8 | 42.0 | 33.9 | 27.4 | 20.6 | 13.9 |
| Volume, cc* | | | | | | |
| Anterior ⅔ | 25.1 | 23.5 | 19.0 | 15.3 | 11.5 | 7.8 |
| Fill Volume | 6.2 | 5.9 | 4.8 | 4.0 | 3.0 | 2.1 |
| Intact, cc** | | | | | | |
| 20% Collapse | 5.0 | 4.7 | 3.8 | 3.2 | 2.4 | 1.7 |
| 30% Collapse | 4.4 | 4.1 | 3.3 | 2.8 | 2.1 | 1.4 |
| 40% Collapse | 3.7 | 3.5 | 2.9 | 2.4 | 1.8 | 1.2 |
| 50% Collapse | 3.1 | 2.9 | 2.4 | 2.0 | 1.5 | 1.0 |
| 60% Collapse | 2.5 | 2.4 | 1.9 | 1.6 | 1.2 | 0.8 |
| 70% Collapse | 1.9 | 1.8 | 1.4 | 1.2 | 0.9 | 0.6 |

Note:
All volumes in cc
*(Belkoff and Molloy 2003; Molloy, Mathis et al. 2003).
**Intact is anterior ⅔ less vertebral shell, cancellous bone 20%, and residual marrow 50%. Calculations assume that 50% marrow will remain after injection and 20% of the volume is occupied by cancellous bone and thus unavailable for filling. Orthovita Inc. (Malvern, PA) www.orthovita.com

Mechanism of Action and Pharmacokinetics of BCNU

BCNU acts as a cell-cycle phase nonspecific antineoplastic agent with alkylating effects on nucleic acids to crosslink DNA strands and prevent cell replication. It is not cross-resistant with other alkylators. As with other nitrosoureas, it may also inhibit several key enzymatic processes by carbamoylation of amino acids in proteins (Mitchell and Schein, 1992.

Intravenously administered BCNU in humans found evidence of rapid plasma clearance and serum protein catalyzed degradation that resulted in little intact BCNU in serum after 60 minutes (Levin et al., 1978). However, in studies with C¹⁴-labeled drug, prolonged levels of the isotope were detected in the plasma and tissue, representing cell-bound radioactive fragments of the parent compound (Levin et al., 1978). The antineoplastic and toxic activities of BCNU are believed to be due to its biotransformation products.

Approximately 60% to 70% of a total dose is excreted in the urine in 96 hours and about 10% as respiratory CO₂ (DeVita et al., 1967). The fate of the remainder is undetermined. Because of the high lipid solubility and the relative lack of ionization at physiological pH, BCNU crosses the blood-brain barrier quite effectively (Levin, 1980). Levels of radioactivity in the CSF are ≈50% of those measured concurrently in plasma.

Efficacy of Intratumoral BEI Mixtures

A single stereotactic intratumoral injection of BCNU-ethanol mixture (67 mg/ml) administered at 5 mg/kg cured 41% of rats (23 cured of 56 treated) bearing well established intracerebral T9 gliosarcoma tumors and produced an increase in life span of 433% for the group, cured animals died of old age (U.S. Pat. Nos. 5,051,257 and 5,162,115). In contrast, the same dose of BCNU delivered systemically produced no cures and no increase in life span. Intratumoral injection of the ethanol vehicle alone produced efficacy in one of thirteen animals.

Intratumoral injection of a BCNU-ethanol mixture (1.5-300 mg/ml) has also been studied in 0.5-1.5 gram Walker Carcinosarcoma 256 tumors growing subcutaneously in rats. It produced extended growth delay in most tumors with many animals becoming long term tumor free survivors. The BCNU-ethanol mixture was active at doses of 2.5, 5 and 10 mg BCNU corresponding to 1.70-20 mg BCNU/gm tumor, and volumes ranging from 5%-100% of the tumor volume (D. Pietronigro, unpublished data, 2004).

Systemic administration of BCNU at the same dose (25 mg/kg) produced no therapeutic benefit. BCNU when formulated in aqueous vehicle and injected intratumorally had antitumor efficacy, but far less than the same BCNU dose formulated in ethanol. Intratumoral injection of the ethanol vehicle alone in volumes of 50%, 75% and 100% of the tumor volume was effective in a few tumors but overall exhibited minimal antitumor effect.

Toxicity of BCNU-ethanol Mixtures in Animal Models

Aside from intravenous dosing studies, the toxicity of BCNU-ethanol mixtures has been studied in normal rat and cat brain, intracerebral rodent tumors, and subcutaneous rodent tumors. The maximally tolerated volume of ethanol that produced no apparent neurologic deficit in rat brain was 20 μl, which extrapolates to 10.8 ml injected into normal human brain. This conversion is based upon comparison of the mass of the rat brain (approximately 2.5 gms) and human brain (approximately 1350 gms).

The maximally tolerated volume of ethanol that produced no apparent neurologic deficit in cat brain was 0.4 ml, which extrapolates to 27 ml injected locally into normal human brain (unpublished data). This conversion is based upon comparison of the cat brain (approximately 20 gms) and human brain (approximately 1350 gms).

Previous Human Use of BCNU in Intravenous Infusions

Nitrosoureas such as BCNU have been used as palliative therapies as a single agent or in combination with other chemotherapeutic agents for the treatment of high- and mid-grade gliomas, lymphomas, small cell lung carcinoma, gastrointestinal cancer, and other carcinomas (Mitchell and Schein, 1992; Carter, 1973; Slavik, 1976). While modest activity has been seen as a single agent in the treatment of many adenocarcinomas, the inability to treat at high doses and more frequent intervals of 6-8 weeks has hampered its clinical efficacy (Carter, 1973; Teicher et al., 1989; Frei et al., 1988).

The usual intravenous dose of BCNU as a single agent in previously untreated patients is 150 to 240 mg/m² every 6 weeks. This is given as a single dose or divided into daily injections such as 75 to 100 mg/m² on 2 successive days, or 80 mg/m² for 3 consecutive days. When BCNU is used in combination with other myelosuppressive drugs or in patients in whom bone marrow reserve is depleted, the doses are adjusted accordingly.

The most frequent and most serious toxicity of BCNU is delayed myelosuppression. It usually occurs 4 to 6 weeks after drug administration and is dose related. Thrombocytopenia occurs at about 4 weeks post-administration and persists for 1 to 2 weeks. Leucopenia occurs at 5 to 6 weeks after a dose of BCNU and persists for 1 to 2 weeks. Thrombocytopenia is generally more severe than leucopenia. However, both may be dose-limiting toxicities. BCNU may produce cumulative myelosuppression, manifested by more depressed indices or longer duration of suppression after repeated doses. Anemia also occurs, but is less frequent and less severe than thrombocytopenia or leucopenia.

Iodinated Contrast Agents

Distribution of the solution within the tumor and the effects of the active agent upon the tissue can be monitored with the use of an imaging method such as ultrasound, MRI (magnetic resonance imaging), or CT (computed tomography). Contrast agents can also be included for use with the appropriate imaging devices including ultrasound, CT, MRI, and PET (positron emission tomography), provided they are compatible with the solvents and active agents used in the invention. The inventors have discovered that preferred contrast agents are mono- or di-iodinated contrast agents that are soluble in alcohol, DMSO or alcohol/DMSO and have a molecular weight of less than 500, preferably less than 450 and even more preferably less than 400. Exemplary iodinated contrast agents includes iodinated imides (e.g., succinamides), purines and pyrimadines; iodinated benzyls, phenols or benzoic acids; iodinated N-pyridones and iodinated glycerols. Non-limiting examples include iodinated succinamide, iodinated glycerol, iodouracil or iofetamine, with iodouracil (such as 5-iodouracil) being particularly preferred.

The inventors have discovered that the size (molecular weight), degree of iodination and lack of charge at physiologic pH are important considerations when selecting an appropriate iodinated contrast agent. The reason is that larger molecular weight agents, for example, those larger than about 500, those that are tri-iodinated or greater and those that carry a charge at physiological pH are entrained in the alcohol solvent only poorly and thus do not permit ready visualization of the course through the needle used to intratumorially inject the solvent/drug solution or dissemination of the solution through the tumor. Thus, the inventors believe that it is particularly preferred to use agents that are generally soluble in alcohol (preferably ethanol), DMSO or alcohol/DMSO mixtures, agents that are di- or preferably mono-iodinated, those that have a molecular weight of less than 500 (preferably less than 450 and more preferably less than 400) and those that are generally non-ionized at physiological pH.

Iofetamine, N-isopropyl-p-iodoamphetamine, has a 335.7 MW as the HCl salt and 301 as the non-salt form (Winchell et al., 1980; Baldwin and Wu, 1988). Following systemic administration, metabolism of iofetamine HCl proceeds sequentially from the N-isopropyl group on the amphetamine side chain. The first step, dealkylation to the primary amine p-iodoamphetamine, occurs readily in the brain, lungs, and liver. The rate-limiting step appears to be deamination to give the transitory intermediate p-iodophenylacetone, which is rapidly degraded to p-iodobenzoic acid and conjugated with glycine in the liver to give the end product of metabolism, p-iodohippuric acid, which is excreted through the kidneys in the urine (Baldwin and Wu, 1988; Druckenbrod et al., 1989).

The compound is lipophilic and crosses the intact blood-brain barrier (Holman et al., 1983; Royal et al., 1985). The plasma pharmacokinetics have demonstrated biphasic elimination half-times of 1.6±1.2 hours and 10.9±6.1 hours (Satoh et al., 1991). Clinically, iofetamine radiolabeled with ¹²³I can be used in brain imaging for lacunar stroke, dementia, and seizures (Druckenbrod et al., 1989; Johnson et al., 1988) and for tumor localization (Nagel et al., 1988; Shinoda et al., 2003) and even to diagnosis pulmonary diseases (Nakajo et al., 1988).

Using phantoms we determined the CT density of ethanol and various concentration of iohexol from 2% to 10%. The linear fit yielded the following relationship: Hounsfield units=68X−217, where X is % iohexol (r²=0.999). Since the 5% iohexol in ethanol (v/v) yielded about 125 Hounsfield units of density and iohexol contains iodides per drug molecule, the inventors believe that the mono-iodinated iofetamine will, at 4% to 5%, be optimal for demarcating vertebral tumors after intratumoral injection.

5-Iodouracil (IUra), 2,4-dihydroxy-5-iodopyrimidine (CAS Registry Number: 696-07-1) has a molecular weight of 238 daltons. The biodistribution literature on IUra is coupled to 5-iodo-2′-deoxyuridine (IdUrd) since IUra is a biotransformation product observed after intravenous infusion of IdUrd in patients (Klecker et al., 1985; Kinsella et al., 1988). These authors observed that following infusion of 500 to1200 mg/m² of IdUrd that the maximum plasma IUra concentration was 100 mumol/L, or about 10 times the simultaneous IdUrd plasma concentration. During the infusion there was at least a fifty- to 100-fold increase in uracil and thymine plasma concentrations. After the infusion, IUra disappearance from plasma was nonlinear, with an apparent Michaelis constant of 30 mumol/L.

With respect to toxicology, there is no direct human toxicology information is available on IUra from direct administration, however, as it is a major biotransformation product of IdUrD and there have been clinical trials with IdUrD at administered doses 100-fold greater than proposed in this trial, it is safe to assume that IUra administered with BCNU-ethanol-DMSO should not be associated with significant myelotoxicity (Kinsella et al., 1988). It is known that IUra is a competitive inhibitor of dihydrouracil dehydrogenase, although when an in vitro human bone marrow assay was used to determine the relative toxicity of IdUrd and IUra it was found that even though exposure to IUra was tenfold higher than that to IdUrd, IdUrd was at least 100 times more cytotoxic to marrow cells (Klecker et al., 1985). The concentration of IUra will be 0.126 mmol/ml for the BEI mixture.

Starting Dose Rationale for BCNU-Ethanol-Iodide Contrast Agent Mixture (BEI)

It has been established that the maximum tolerated dose of the BCUN-ethanol mixture for the intratumoral treatment of glioblastoma is 240 mg BCNU and 5 ml absolute ethanol (Hassenbusch et al., 2003). This equates to a concentration of 48 mg BCNU/ml absolute ethanol. Unlike the cerebral glioblastoma tumors treated in that study, where the adjacent normal brain was considered to be a substantial morbidity risk, the intratumoral injection of vertebral metastases should poise a much lower normal tissue toxicity risk. The inventors thus contemplate injections of a BEI mixture with BCNU at a concentration of about 20 mg/ml and an injection volume of 50% of the computed tumor volume will be preferred. However, various ranges of BCNU administration are contemplated and include a preferred, non-limiting, concentration range of 20 mg/ml ethanol to 35 mg/ml ethanol at volumes of injection ranging from 0.5 ml to 4 ml of the mixture, depending on tumor size.

Intratumoral Therapy

The invention provides, in certain embodiments, intratumoral injection of formulations of the present invention (with or without the use of a contrast agent) to treat spinal metastases. Although it may be desirable, in certain embodiments, to deliver BCNU-ethanol extratumorally, intratumoral injection can be used to effectively deliver BCNU to the tumor.

These formulations, particularly BCNU-ethanol mixtures, allow direct intratumoral injection of, e.g., BCNU in a formulation designed to rapidly deliver it throughout the tumor in high doses. Because of its delivery directly into the tumor, smaller total doses of BCNU can be administered compared to those needed intravenously. At the same time, this lower dose produces much higher intratumoral doses of BCNU and is more effective than intravenous BCNU as demonstrated in two animal tumor models (U.S. Pat. Nos. 5,051,257 and 5,162,115).

Intratumoral injection has several advantages. This approach has the potential to deliver the highest dose of antineoplastic agent directly into tumors while producing the lowest extratumoral dose. Intratumoral chemotherapy for brain tumors tumors has been administered by direct injection, intracavitary instillation, intracavitary topical application, chronic low-flow microinfusion and controlled release from polymer implants (Bosch et al., 1980; Bouvier et al., 1987a; Bouvier et al., 1987b; Brem et al., 1991; Garfield and Dayan, 1973; Garfield et al., 1975; Kroin and Penn, 1982; Livraghi et al., 1986; Penn et al., 1983; Ringkjob, 1968; Rubin et al., 1966)). However, the efficacy of intratumoral chemotherapy has been limited by the inability to effectively distribute drug throughout the tumor (Garfield et al., 1975; Kroin and Penn, 1982; Sendelbeck and Girdis, 1985; Morrison and Dedrick, 1986).

The intratumoral methods tested to date are similar in that they all formulate antineoplastic agents into vehicles, either liquids (aqueous or oil) or solids, which themselves are incapable of tumor penetration. These vehicles act simply as depots that introduce the agent into the tumor site. The ability of antineoplastic agents to diffuse through tumors is restricted and tissue clearance by permeation and reaction is substantial (Kroin and Penn, 1982; Sendelbeck and Girdis, 1985; Morrison and Dedrick, 1986). These factors result in a rapid decrease in drug concentration as a function of distance from the depot (Kroin and Penn, 1982; Sendelbeck and Girdis, 1985; Morrison and Dedrick, 1986).

A method for specifically formulating antineoplastic agents into solutions capable of rapidly perfusing tumors has been reported (U.S. Pat. Nos. 5,051,257 and 5,162,115). Direct delivery organic solvent vehicles which are water miscible but also easily move through biological cell membranes has been injected directly into tumors where it has been shown to rapidly penetrate the tumor and transports the antineoplastic agent through the tumor. To date, the BCNU-ethanol mixture, composed of the antineoplastic agent BCNU [1,3-bis(2-chloroethyl)-l-nitrosourea] dissolved in absolute ethanol, has been most widely used for this purpose. The ability of ethanol to readily permeate tumors following its direct intratumoral injection has been established in humans (Livraghi 1993).

In certain embodiments a composition comprising a chemotherapeutic, an alcohol and an iodinated contrast agent may be delivered to a cancerous or pre-cancerous tumor. For example, the tumor may be liver metastases. Injections of alcohol (ethanol) have also been used for the treatment of liver metastases from a variety of primary hepatic tumors, colorectal carcinoma, and carcinoid/neuroendocrine tumors (Kessler et al., 2002; Erce and Parks, 2003; Garcea et al., 2003; Kurokohchi et al. 2004; Atwell et al., 2005; Jain et al., 2005). The composition may also be used for the treatment of metastases to retroperitoneal lymph nodes (Zuo et al., 2004) and/or even lymph nodes in the neck or pelvis.

It is anticipated that use of an iodinated contrast media with the present invention may allow for improved treatment of urologic tumors. Previously, treatment of urologic tumors has been limited by poor biodistribution (Rehman et al., 2003), suggesting that it was too difficult to follow and/or define the injection site. It is envisioned that the addition of iodinated contrast would quickly improve that limitation. The present invention may also be used to control vertebral hemangioma (Doppman et al., 2000; Bas et al., 2001; Murugan et al., 2002).

The present invention may be used with radiofrequency or cryoablation. Radiofrequency with ethanol injections appear to work better than either alone (Siperstein and Berber, 2001; Shankar et al., 2004). Similar results have also been observed in combination with cryoablation (Xu et al., 2003).

Additionally, the present invention may also be used to treat non-cancerous tumors. Alcohol injections have been used in the treatment of uterine chorioangioma (Wanapirak et al., 2002) and the addition of contrast might also improve extent and proper localization of the injection. It is envisioned that an iodinated contrast media may be added to an alcohol solution to aid in injection, and, in certain embodiments, it may be desirable to exclude chemotherapeutics and/or cytotoxic drugs from the solution.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Formulary Preparation of BEI

Preferred pharmaceutical preparations of BEI (BCNU, 5-iodouracilo, DMSO, and absolute ethanol) are typically prepared to achieve BCNU at 20 mg/ml and 5-iodouracil at 29 mg/ml as follows:

145mg of 5-iodouracil powder is weighed out using a prescription balance, and placed in a 5 ml sterile empty vial, Using a filter straw, 1 ml sterile DMSO is withdrawn into a 5ml syringe and added to the 5-iodouracil powder. This mixture is gently swirled to dissolve the powder. Then, using a filter straw, 5 ml sterile ethyl (Absolute) alcohol is drawn into a 10 ml syringe, and 4 ml of the alcohol from the syringe is added to the DMSO-iodouracil mixture. This mixture is then again gently swirled to confirm that the iodouracil remains dissolved and the DMSO mixes with the ethanol. Next, the 5-iodouracil solution is drawn into a new 10 ml syringe using a new filter straw, and the volume is QS in the syringe to 5ml using the absolute alcohol remaining in the original syringe.

A Millex GV 0.22 micron filter is added to the syringe containing the 5-iodouracil solution, and it is filtered into a 100 mg vial of commercial BCNU (Carmustine), and the mixture swirled gently to dissolve BCNU and to combine the 2 products. The top of the BCNU vial is wiped, sealed with an IVA Pressure Sensitive Security Seal and labeled appropriate labeling.

### Example 2

### Treatment of Vertebral Metastases With BEI

This example sets forth proposed clinical trials that demonstrate the clinical application of BEI. In particular, described are protocols to make preliminary assessment of the safety and efficacy of the direct tumor injection of the BEI formulation for the treatment of inoperable vertebral metastases from carcinoma of breast, lung, or prostate or renal cell carcinoma. This example further explains how to determine, using computerized tomography (CT) guided intratumoral injection, the optimum dose of BCNU, volume of absolute ethanol, and amount of nonionic contrast needed to achieve optimal BCNU dose delivery to intravertebral metastatic carcinomas, to determine the qualitative and quantitative toxicity of BCNU-ethanol-nonionic contrast mixture administered by intratumoral injection and to assess the therapeutic activity of the BEI mixture in patients with inoperable vertebral metastases from carcinoma of breast, lung, or prostate or renal cell carcinoma.

In this protocol the inventors propose to inject vertebral metastases with the BEI a mixture of 1,3-bis(2-chloroethyl)-1-nitrosourea (BCNU) in ethanol prepared as described in Example 1. A similar therapeutic mixture (which did not contain the contrast agent) has been studied as DTI-015, a proprietary preparation of Direct Therapeutics Incorporated. DTI-015 has been extensively studied as an intratumoral treatment for recurrent glioblastoma and other high-grade gliomas (Hassenbusch et al., 2003). The direct injection of BCNU-ethanol mixture into experimental tumors produces a 100- to 1000-fold increase in tumor BCNU over that attainable by intravenous administration (Hamstra et al., 2005). BCNU exerts part of its clinical activity through DNA alkylation and cross-linking. Bodell and colleagues found that they could measure N7-(2-hydroxyethyl)guanine (N7-HOEtG), one of the DNA alkylation products formed by BCNU, in experimental tumors and glioblastoma tumors at the time of surgery (Bodell et al., 2001; Bodell et al., 2003). As an example, they found levels of N7-HOEtG detected in RIF-1 tumors following intra-tumoral administration of BCNU-ethanol mixture were 164-fold higher than levels of N7-HOEtG in the intra-peritoneal BCNU treated tumor samples (Bodell et al., 2003; Bodell et al., 2007).

In the phase I study of BCNU-ethanol mixture in glioblastoma patients, it was found that the maximum tolerated dose (MTD) was 5 ml of ethanol and 240 mg (Hassenbusch et al., 2003). Based on fill volume estimates published for vertebral bodies, it is highly likely that vertebral tumors will occupy less than 6 cc/vertebra (Table 1). If that in fact is the case, then injecting BCNU-ethanol mixture at volumes between 2 and 6 cc may be feasible and therapeutic. Because of this belief, the inventors propose a phase I/II study to determine an optimal ethanol volume and BCNU amount that will be safe for patients and, at the same time to evaluate whether intratumoral injection of BCNU-ethanol mixture can control vertebral tumor growth and alleviate symptoms such as pain, weakness, sensory loss, and bladder or bowel sphincter dysfunction.

Proposed Clinical Study Details

Patient Eligibility

This study will accrue approximately 25 patients. Inclusions: Patients must be between the ages of 18 and 75, inclusive. Patients will be selected from those seen by the medical oncology services at the University of Texas M. D. Anderson Cancer Center UT MDACC). Patients must have histologic proof of an extra-vertebral primary carcinoma (breast, lung, prostate, renal cell).

Patients must have radiographic evidence that unequivocally supports a diagnosis of vertebral metastasis and, based on CT, the tumor will have less than 75 Hounsfield units of intensity.

Patients must have a biopsy (may be immediately prior to administration of BEI) that confirms the diagnosis of vertebral metastasis or radiographic evidence that supports no other reasonable diagnosis.

There must be a tumor volume of each tumor component between 0.5 and 6 cm³ (equates to a diameter of less than 2.25 cm). The patient must have a Kamofsky functional status rating ≧60 (See Appendix B). Patients must be fully recovered from the acute effects of any prior chemotherapy or radiotherapy.

Patients must be able to read and understand the informed consent document and must sign the informed consent indicating that they are aware of the investigational nature of this study in keeping with the policies of this hospital. The only acceptable consent form is attached at the end of this protocol.

For females of childbearing potential (pre-menopausal without a history of a sterility operation), the patient must not be pregnant as evidenced by a menses in the last 8 weeks or by a negative urine HCG pregnancy test.

Patient Exclusions

Tumor to be injected is in a partially collapsed or fibrotic vertebra. Radiotherapy to the specific vertebral metastasis within 6 weeks of this study. Patients with active uncontrolled infection. Serious liver or bone marrow disorder—specifically serum bilirubin>2.0 mg %, SGOT>2.5× normal, SGPT>2.5×, absolute neutrophil count<1500/mm³, platelet count<100,000/mm³. Evidence of a bleeding diathesis or use of anticoagulant medication. Inability to obtain informed consent because of psychiatric problems or complicating medical problems which render the patient ineligible. Unstable or severe intercurrent medical conditions. For females: pregnancy, risk of pregnancy (i.e., unwillingness to use adequate protection to prevent pregnancy), breast feeding a baby during the study period, or lactation. Neurologic compromise requiring treatment with radiation therapy or surgery.

Treatment Plan

Study Overview

The preliminary aspect of this trial of BEI will be conducted in patients with vertebral metastases from carcinoma. Patients undergoing a biopsy will be biopsied using standard CT imaged guided localization procedures initially although later patients may be treated under fluoroscopy. The results of this study are intended to provide the basis for an initial evaluation of the safety, tolerability, and feasibility of administering BEI as an intratumoral injection and treatment for patients with vertebral metastases from carcinoma. The study will involve a maximum of 25 patients.

Volume escalation: BEI will be administered by a single free-hand intratumoral injection in a volume to fully distribute in the tumor and at a BCNU concentration of 20 mg/ml. The volume (in ml) of BEI will be calculated as a percentage of the tumor volume. The starting dose will be 50% of the calculated tumor volume with one escalation to 75% of tumor volume. The dose of BCNU will be fixed at a concentration of 20 mg BCNU/ml ethanol-iohexol. The maximum volume to be administered will be 4 ml.

Dose escalation: If 3 consecutive patients at the 75% dose level show no or minimal toxicity, the BCNU concentration will be increased to 30 mg/ml. No further dose escalations will be studied. Based on direct injection of glioblastoma tumors at doses as high as 48 mg/ml ethanol (Hassenbusch et al., 2003) the inventors do not expect to see appreciable systemic myelotoxicity from one intravertebral tumor injection with BEI.

Each patient will receive a single injection, with at least three patients being treated at each dose level. Doses may be escalated after two patients have shown no or minimal (NCI toxicity grade 1 or 2) toxicity for at least 2 weeks after the injection. Dose escalation will be halted when the maximum tolerated dose (MTD) has been reached. The MTD will be defined as the highest dose level at which two patients show no or minimal (NCI toxicity grade 1 or 2) toxicity. Once the MTD is established, three additional patients will receive this dose level for a total of six patients at the MTD level. The recommended Phase II dose will be the dose that the last 3 patients receive. Table 2 below summarizes the plan. Note that for poor injections or some types of toxicity, patient cohorts may increase to up to 6 patients per treatment cohort.

TABLE 2
Basic aspects of patient cohorts, BCNU
concentration, and ethanol injected.

| | Patient | BCNU, mg/ml | Percentage | Ethanol |
|---|---|---|---|---|
| Cohort | numbers | ethanol | of tumor volume | volume, ml |
| 1 | 3 | 20 | 50% | <4 ml |
| 2 | 3 | 20 | 75% | <4 ml |
| 3 | 0 to 3 | 30 | 75% | <4 ml |

Evaluations prior to enrollment in the study and after injection of BEI include a complete history and physical, weight, recording of steroid use, functional status evaluation, and quality of life evaluation, full neurological evaluation, vital signs and CT and MRI spine scans. Laboratory studies will include CBC, differential, platelet count, PT, PTT, and chemistry profile and electrolytes, and pregnancy test (in childbearing potential women). A follow-up CT scan will be obtained 2 hrs after administration of BEI and MRI will be obtained at 4 and 8 weeks following treatment. Toxicities will be evaluated according to the NCI Common Toxicity Grading Criteria.

Diagnostic Imaging Technique: Patients to be recruited for this protocol will initially be identified on MRI or CT of the spine. Spinal lesions suspicious for malignancy and able to biopsied and injected with BEI will be identified.

Selected patients will undergo a planning CT study to help guide the image-guided biopsy. This will be performed using a modified contrast enhanced CT of the spine utilizing a routine non-contrast high resolution scanning (1.25 mm collimation, pitch of 1.375) using a multidetector helical CT of the spine segment in question (typically either the thoracic or the lumbar spine). The non-contrast scan will be followed by a bolus of radiographic contrast material (administered through a peripheral IV at 4 cc/second) and subsequent contrast enhanced imaging. Contrast enhanced imaging will be performed at multiple time phases through the level to be biopsied using standard high-resolution helical technique (Table 3). Only the level to be biopsied (for example, the L4 vertebra, a 12 cm Z-direction volume, estimated scan time 9 seconds) will be scanned at 25 seconds after contrast with successive subsequent delays of 15, 20, 30, and 60 seconds after each successive image acquisition (this type of CT will henceforth be referred to as a 4D CT).

TABLE 3
Timing of CT based on high-resolution helical technique.

| | | Delay after | | | | | |
|---|---|---|---|---|---|---|---|
| | | contrast | Delay after previous scan | | | | |
| | Pre-contrast | 25 | 15 | 20 | 30 | 30 | 60 |
| | Spinal | Level of | Level of | Level of | Level of | Level of | Level of |
| Volume Imaged | Segment | Lesion | Lesion | Lesion | Lesion | Lesion | Lesion |
| Cumulative Time | | 25 | 25 + 9 + 15 | 25 + 9 + 15 + | 25 + 9 + 15 + | 25 + 9 + 15 + | 25 + 9 + 15 + |
| after Start of | | | | 9 + 20 | 9 + 20 + 9 + 30 | 9 + 20 + 9 + | 9 + 20 + 9 + 30 + |
| Bolus (s) | | | | | | 30 + 9 + 30 | 9 + 30 + 9 + 60 |
| Total (s) | Pre | 25 | 49 | 78 | 117 | 156 | 225 |

There will be non-contrast CT of the whole spinal segment, with 6 successive contrast enhanced images through the level of interest at various time points in the distribution of the intravenous contrast bolus. This is a technique similar to our current practice in other body parts such as the neck, and is well described in the literature (Izzo et al., 2005; Goh et al., 2005; Seo et al., 2005; Li et al., 2005; Goh et al., 2005; Wintermark et al., 2005). These images will be post processed to extract perfusion data and subjected to neuroradiological interpretation. Using this information, the patient will be reassessed in terms of the safety and feasibility of biopsy and potential injection of ethanol-iohexol or BEI into the vertebral tumor. If still deemed a suitable candidate, the patient will be offered the opportunity to participate in this study, or alternatively another means of treatment will be offered as clinically indicated.

Potential reasons to exclude a lesion at this stage would include a reasonable suspicion that the injection agent(s) might come into direct contact with the nerves or spinal canal contents, lesion anatomy unfavorable to a safe needle approach, or lesions suspected to be too vascular to biopsy safely.

Lesion volumetrics will be performed by the Computational Neurolmaging lab using either the CT or MR data, using the data set best suited as indicated by the supervising neuroradiologist.

Image Guided Biopsy

Biopsy will be performed as per the routine radiologic practice utilizing sterile technique and CT image guidance. Supervised conscious sedation will be administered by an anesthesiologist. The vertebra previously identified as the level of abnormality will be identified on scanogram images, and a focused CT will be performed through that level using high-resolution technique similar to that employed during the planning CT examination. The neuroradiologist or neurosurgeon performing the procedure will identify an appropriate approach to the lesion, which could be either transpedicular or parapedicular as the morphology and location of the lesion dictates. CT guidance will be used to insert a bone biopsy needle system through the skin and underlying tissues to the bone overlying the target lesion. A coaxial biopsy system which will allow the secure positioning of an outside guiding cannula with the exchange of coaxial central biopsy devices will be used (there are several types of suitable commercially available devices in routine use). The biopsy device will be placed with the outer guiding cannula at the periphery of the lesion, just beyond the bony covering of the lesion. Fine needle aspiration of the lesion will then be performed using multiple passes with a needle through the substance of the lesion itself. Onsite cytological examination of the aspirates will be performed and the adequacy of the cytology specimen will be determined. Once adequate sampling has been obtained and the malignant nature of the lesion confirmed, the inventors will proceed to perform injection of BEI into the tumor.

Using the same coaxial cannula already in place, a needle will be placed with its tip centrally within the lesion itself. The previously calculated volume of BEI will then slowly be injected through the needle under imaging guidance. After every 0.3-1 cc of injections, a CT scan will be performed through the limited area covering the lesion, to assess the distribution of BEI through the lesion matrix. If necessary, the needle will be repositioned to achieve adequate spread of BEI through the substance of the tumor. Once the entire volume of BEI has been injected, a final CT scan will be performed to document the distribution of the agent. At this point, the needle will be withdrawn along with the coaxial guiding cannula, the sedation will be terminated and the patient will be brought to recovery.

As part of the post procedure monitoring of the patient, repeat targeted non-contrast CT of the treated level will be performed at 2 hours after BEI to document the iohexol dye distribution in tumor.

The patient will be observed until completely recovered from the sedation and will then be released to the care of the treating physician.

A repeat 4D CT and MRI study of the spinal segment in question will be performed about 24 hours after the procedure.

Management of Patient for Injection of BEI

Handling of drug and infusion solution: The BCNU drug product (lyophilized powder) should be kept refrigerated in a light-protected container and is stable for at least 1 year under these conditions. The 5-iodouracil (powder) should be stored in a light-protected container at room temperature and is stable for 1 year under these conditions.

Preparation of infusion solution: Immediately (within 2 hours) before injection of BEI, the final solution will be prepared. The solution will be prepared as described in Example 1. The solution will be placed in a syringe and protected from light by placing in a brown amber bag. BCNU has been found to be stable, even in pure water without any ethanol, for 46 hours at room temperature when shielded from light. It is planned to use the BEI within 4 hours of its preparation.

Pretreatment Evaluation

History: general medical history with emphasis on neurologic and musculoskeletal symptoms.

Medication: all drugs being taken will be recorded including glucocorticoids and pain medicine at the time of patient accrual to the study.

Physical examination: General examination and detailed neurological examination. Vital signs, weight, and BSA will also be recorded.

Radiologic evaluation: see section 5.2 for details. The baseline MRI scan will be electronically scanned, the images digitized, and tumor outlined electronically on each slice of either a coronal plane or an axial plane series. Using standard computer software, the tumor will be reconstructed three-dimensionally and the tumor volume will be calculated from the area of tumor on each slice, the slice thickness, and the space between slices. A non-contrast CT will be required to determine if the tumor to be injected has less than 75 Hounsfield units of intensity so the BEI distribution can be determined post-injection.

Quality of life assessments to be completed on the day or day before the injection BEI (before pre-medication).

The Brief Pain Inventory (BPI; Appendix C) is a refinement of the Wisconsin Brief Pain Questionnaire (Daut et al., 1983) that was developed by Cleeland and Daut for use mainly in cancer patients, though it has been found to be valid and reliable in non-cancer patients as well. Over 1,200 cancer patients with cancer at four anatomical sites (breast, prostrate, colorectal and gynecological) were used during the instrument's development phase. The BPI is an ordinal multidimensional scale consisting of a 20-item questionnaire that assesses pain history, intensity, location, quality, interference with activities, and cause. It is self-administered and requires approximately 15 minutes to complete. Intensity is recorded on a numerical scale ranging from 0 to 10 (0 meaning no pain and 10 meaning pain as bad as you can imagine). As pain may vary during a given time period, be it day or week, the intensity is rated at the time of completing the questionnaire, and also at its worst, least, and average over the past day or week, depending on the context of the study. Final intensity scores may represent the worst or the average pain. The impact of the pain is represented as a mean of 6 scores used to indicate level of pain, interference with mood, walking, other physical activity, work, relationships with others, and sleep. Each score can range from 0 or “no interference” to 10 or “interferes completely”. The location of pain is indicated on a diagram of a human figure. The patients are asked to choose from a list of words (taken from the MPQ) the word that best describes their pain and to indicate the extent and duration of pain, and any relief secondary to analgesics. Evidence of the reliability and validity of the BPI is available.

The problematic issue of the multitude of pain assessments in the medical literature was recently addressed by an expert panel of pain experts, the IMMPACT (the Initiative on Methods, Measurement, and Pain Assessment in Clinical Trials) group and reported in the pain literature (Dworkin et al., 2005; McQuay, 2005). One of the problems with the multitude of available instruments is selecting the appropriate one(s) for the clinical situation (Wittink et al., 2004).

Based on the IMMPACT group recommendations, assessment should be carried out in 6 core outcome domains: pain, physical functioning, emotional functioning, participant ratings of improvement and satisfaction with therapy, symptoms, adverse events, and participant disposition. The article further delineates recommended scales for each domain. For pain, a 0-10 scale is recommended with analgesic use recorded. Physical functioning is assessed by the Multidimensional Pain Inventory Interference Scale or the BPI pain interference items. Emotional functioning can be assessed by the Beck depression inventory. Participant ratings of improvement can be assessed by the Patient Global Impression of Change scale. Adverse event reporting and disposition should include compliance with treatment and reasons for withdrawal from treatment.

Additional baseline tests will include the following: Functional Independence Measure (Weitzner et al., 1995) and Kamofsky Functional Status Evaluation (see Appendix B).

Laboratory testing: Blood Chemistry: Alanine aminotransferase (SGPT), Albumin, Aspartate aminotransferase (AST), Alkaline phosphatase, Bilirubin (total and direct), BUN, Calcium, Creatinine, Glucose, Phosphorus, Potassium, and Total protein, . Hematology: Differential Count, Hematocrit, Hemoglobin, Platelet Count, Protime (PT), Partial Thromboplastin Time (PTT), White Blood Cell Count (WBC), Absolute Neutrophil Count (ANC).

Evaluation During Study

After injection of BEI, daily assessments will be done for pain scores (from BPI; Appendix C) for 7 days and then weekly for 2 months. In addition, patients will record other symptoms and daily medication use with emphasis on pain medication, glucocorticoids, and psychoactive agents.

History: At 4 and 8 weeks after BEI injection should include evaluation for back, leg, and arm pain, urinary retention or incontinence, arm/leg weakness, arm/leg/trunk paresthesia and/or dysesethesia.

Physical Examination, including neurologic examination: General physical examination at 8 weeks after injection and neurological examination at 4 and 8 weeks after BEI injection.

Functional neurologic status and quality of life assessment: The tests in section 6.5 will be completed at 4 and 8 weeks, while pain scales will be obtained daily for 8 weeks.

Laboratory Testing: At 4 and 8 weeks after injection: differential count, hematocrit, hemoglobin, platelet count, red blood cell count (RBC), and white blood cell count (WBC). At 8 weeks after injection: SGPT, albumin, aspartate aminotransferase (AST), alkaline phosphatase, bilirubin (total and direct), creatinine, calcium, and uric acid.

Radiologic evaluation: Follow-up of the treated vertebral lesion will be done primarily with serial imaging studies supplemented with clinical exam and appropriate lab testing as required.

A 4D CT of the spine utilizing the same protocol as used in the pre-operative assessment of the lesion will be obtained 2 hrs after the injection of BEI.

MRI with gadolinium enhancement, according to routine departmental protocol, will be at 4 and 8 weeks after the procedure and thereafter as per routine guidelines. Each MRI scan will be electronically scanned, the images digitized, and tumor outlined electronically on each slice of either a coronal plane or an axial plane series. Using standard computer software, the tumor will be reconstructed three-dimensionally and the tumor volume will be calculated from the area of tumor on each slice, the slice thickness, and the space between slices.

Follow up after 2 months: If there is no tumor progression at 8 weeks post-injection, the patient will be followed by the study research nurse until tumor progression or patient death. This follow-up will consist of verbal contact (by phone or in person when the patient comes to clinic for other reasons) every 1-2 months to assess history (as outlined above), pain medication, glucocorticoid use, BPI (Daut and et al., 1983), Functional Independence Measure (Linacre et al., 1994), Karnofsky Functional Status Evaluation (see Appendix B). Results of any follow-up MRI scans and neurologic examinations during this same time period will be recorded.

Loss of follow up: If a patient drops out or is discontinued from the study for any reason, every effort will be made to complete all post-treatment procedures and assessments.

Criteria for Treatment Respinse and Toxicity

Response to BEI will be judged by clinical and radiologic measures. Response will be characterized as clinical, radiologic, or both.

Serial pain and symptom history and medication records will be one measure of therapeutic benefit of BEI treatment. Clinical improvements will dependent on a patient experiencing (a) less pain and dependence on pain medication, (b) improvement in affected limb strength, (c) reduced sensory dysfunction in affected limbs or back, (d) increased mobility, and (e) improved quality of life.

Serial neurologic examination will determine if the patient has objective evidence of increased strength in affected limbs, improved gait, less sensory dysfunction, improved affect, and the like.

Serial MRI with and without gadolinium-enhancement will be used to measure the extent of tumor to determine if the tumor has grown since BEI treatment. Because of possible BEI treatment-induced artifacts, reduction in MRI lesion volume may be more difficult to ascertain than tumor growth (Hassenbusch et al., 2003; Hall et al., 2004).

Toxicity Assessment

Toxicity will be determined acutely for the period during and for 24 hours after BEI injection. Subacute toxicity to BEI will be judged to be toxicity recorded within 2 weeks of BEI. In all cases the focus will be on pain, sensory changes, and weakness in limbs and truck associated with the tumor injected with BEI.

All toxicity will be recorded using the NCI Common Toxicity Grading Criteria (Appendix A) for grading acute and subacute toxicity. Myelosuppressive toxicity shall be reported as lowest observed WBC, absolute neutrophil count (ANC), and platelet count. Anemia and red blood cell transfusions will be noted. Renal and hepatic toxicity will be reported as changes in creatinine, SGPT, LDH, bilirubin, and alkaline phosphatase.

Definition of Adverse Events—one or more of: All changes in the general condition of a patient. Subjective and objective symptoms (spontaneously offered by the patient and/or observed by the investigator or the study nurse). All concomitant diseases which occurred after the start of the clinical trial. All relevant changes in blood chemistry findings during the trial (mainly blood cell count, platelets, liver enzymes, electrolytes, glucose, and creatinine)

Definition of Serious Adverse Events—one or more of: Death of a patient. Life-threatening events. Any clinical experience that is permanently disabling or requires (extended) hospitalization. Induction of new cancer. Overdose.

Definition of Unexpected Adverse Events An AE is considered an unexpected AE if it is not mentioned in the Investigator's Brochure, or if it is of greater frequency and/or severity than that mentioned in the Investigator's Brochure. All adverse events will be examined to determine any relationship to the administration of BEI. A possible relationship will be categorized as definite, probable, possible, conditional, or doubtful.

AEs will be recorded on the appropriate forms. Serious AEs or unexpected AEs, and all deaths on study, whether considered to be drug related or not, will be reported by appropriate form on the next business day to the Surveillance Committee (Institutional Review Board) and by telephone to the FDA.

Any serious adverse event or unexpected adverse event, whether or not study drug related, will be reported in detail and in writing to the Surveillance Committee (Institutional Review Board) by the end of the next business day after it occurs and will be reported by written form to the FDA within 10 working days. Any death, whether considered to be drug related or not, and any life-threatening adverse event, considered to be drug related, will be reported in detail and in writing to the Surveillance Committee on the next business day, and to the FDA within three working days.

Criteria for Removal from Study

Progressive disease as defined above. Failure of patient to appear or be available for follow-up evaluations. Patient request to be removed from study. Patient death during study. Need for further antitumor therapy (operation, radiotherapy, and/or other chemotherapy) during the follow-up period. For the purposes of this section, steroids will not be considered as further antitumor therapy. The need for further therapy will usually be based upon progressive disease.

The reason(s) for a patient's removal from study will be clearly documented on Case Report Forms. Should a patient expire during the study, the Investigator will relate autopsy data (if available) to the patient's clinical course, if possible. A copy of the death certificate and autopsy report, if performed, will be provided.

Any patient prematurely removed from the study for any reason will be followed, to the best extent possible, until 12 weeks after the BEI injection. This will provide as much information about this patient as can be physically obtained.

Statistical Information

The goal of this protocol is assess the preliminary toxicity and therapeutic activity of BEI in patients with inoperable vertebral metastases from carcinoma of breast, lung, or prostate or renal cell carcinoma. Based on the volume and dose escalation parameters the inventors would expect to study up to 12 patients to find a safe injection dose and volume. To insure that the dose is safe and has some activity, the inventors would plan to accrue an additional 6 patients at the appropriate study dose.

Efficacy assessment will only involve extremes of tumor response in all patients or in no patient. This assessment of efficacy denotes that, from a statistical viewpoint, in a study of only 9 patients at the study dose, a statistically-valid statement of efficacy or lack of efficacy can be made only if all or almost all patients show a tumor response or if all or almost all patients do not show a tumor response, respectively. Response will have variable names in that the inventors are not certain whether pain, mobility, or radiographic tumor control will be indicate a “successful” outcome to BEI treatment. For that reason, the inventors have included a number of parameters to evaluate for “response” (see above).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,051,257
U.S. Pat. No. 5,162,115
U.S. Pat. No. 6,753,005
Atwell et al., Cardiovasc. Intervent. Radiol., 28(4):409-421, 2005.
Baldwin and Wu, J. Nucl. Med., 29:122-124, 1988.
Bas et al., Spine, 26(14):1577-82, 2001.
Belkoff and Molloy. Spine 28(14): 1555-1559, 2003
Bilsky et al., In: Spine Surgery: techniques, complication avoidance, and management,
Benzel (Ed.), 2^{nd} Edit., Philadelphia: Elsevier/Churchill Livingstone, 1025-1061, 2005.
Bodell et al., J. Neurooncol., 62(3):251-258, 2003.
Bodell et al., Neuro-oncol., 3(4):241-245, 2001.
Bodell et al., Neuro-oncol 9(1):12-19, 2007
Bosch et al., Acta Neurochir. Suppl. (Wien), 30:441-444, 1980.
Bouvier et al., Appl. Neurophysiol., 50(1-6):223-226, 1987.
Bouvier et al., Neurosurgery, 20(2):286-291, 1987.
Brem et al., J. Neurosurg., 74(3):441-446, 1991.
Carter, Cancer Chemother. Rep., 4(3):35-46, 1973.
Chan and Zackheim, Arch. Dermatol., 107(2):298, 1973.
Daut et al., Pain, 17(2):197-210, 1983.
DeVita et al., Clin. Pharmacol. Ther., 8(4):566-577, 1967.
Doppman et al., Radiology, 214(2):341-8, 2000.
Druckenbrod et al., Dicp, 23:19-24, 1989.
Dworkin et al., Pain, 1 13(1-2):9-19, 2005.
Erce and Parks, Br. J. Surg., 90(3):272-289, 2003.
Frankel et al., Paraplegia, 7(3):179-192, 1969.
Frei et al., Cancer Res., 48(22):6417-6423, 1988.
Garcea et al., Eur. J. Cancer, 39(15):2150-2164, 2003.
Garfield and Dayan, J. Neurosurg., 39(3):315-322, 1973.
Garfield et al., Clin. Oncol., 1(3):213-222, 1975.
Gilbert et al., Ann. Neurol., 3(1):40-51, 1978.
Goh et al., Am. J. Roentgenol., 185(1):225-2231, 2005.
Goh et al., J. Comput. Assist. Tomogr., 29(1):59-63, 2005.
Gokaslan et al., J. Neurosurg., 89(4):599-609, 1998.
Hall et al., Clin. Cancer Res., 10(23):7852-7859, 2004.
Hammoud et al., Am. J. Roentgenol., 180:1469-1475, 2003.
Hamstra et al., J. Neurooncol., 73(3):225-238, 2005.
Hassenbusch et al., Neoplasia, 5(1):9-16, 2003.
Holman et al., J. Nucl. Med., 24:922-931, 1983.
International standards for neurological and functional classifications of spinal cord injury. Chicago: American Spinal Injury Association; 1996.
Izzo et al., J. Exp. Clin. Cancer Res., 24(2):209-215, 2005.
Jain et al., Hepatogastroenterology, 52(65):1567-1584, 2005.
Johnson et al., Arch. Neurol., 45:392-396, 1988.
Kessler et al., Isr. Med. Assoc. J., 4(12):1106-1110, 2002.
Kitamura et al., Cancer Res., 56(17):3986-3992, 1996.
Koositra et al., Cancer Res., 49(4):977-982, 1989.
Klecker et al., Clin. Pharm. & Ther., 38: 45-51, 1985
Kinsella et al., J. Clin. Oncol., 6 : 871-879, 1988.
Kroin and Penn, Neurosurgery, 10(3):349-354, 1982.
Kurokohchi et al., Int. J. Oncol., 24(2):381-387, 2004.
Laskar and Ayres, J. Pharm. Sci., 66(8):1076-1078, 1977.
Levin and Levin, Selective Cancer Therapeutics, 5(1):33-35, 1989.
Levin et al., Cancer Chemo. Pharma., 1(4):233-242, 1978.
Levin et al., Cancer Treatment Reports, 62(9):1305-1312, 1978.
Levin, J. Medicinal Chem., 23(6):682-684, 1980.
Li et al., World J. Gastroenterol., 11 (9):1287-1291, 2005.
Linacre et al., Arch. Phys. Med. Rehabil., 75(2):127-132, 1994.
Livraghi et al., Tumori, 72(1):81-87, 1986.
Livraghi, Z Gastroenterol., 31(4):260-264, 1993.
Maranzano and Latini, Int. J. Radiat. Oncol. Biol. Phys., 32(4):959-967, 1995.
Maranzano et al., Cancer, 67(5):1311-1317, 1991.
McQuay, Pain, 113(1-2): 1-2, 2005.
Mitchell and Schein, In: The Chemotherapy Sourcebook, Perry (Ed.,) Williams & Wilkins, Baltimore, 384-397, 1992.
Molloy et al. Spine 28(14):1549-1554, 2003.
Morrison and Dedrick, J. Pharm. Sci., 75(2):120-128, 1986.
Moulopoulos et al., J. Magn. Reson. Imaging, 6(4):667-674, 1996.
Murugan et al., Neurol. India, 50(3):300-305, 2002.
Nagel et al., Arch. Neurol., 45:1126-1128, 1988.
Nakajo et al., Ann. Nucl. Med., 2:49-53, 1988.
Penn et al., Appl. Neurophysiol., 46(1-4):240-244, 1983.
Perrin et al., In: Cancer in the Nervous System, Levin (Ed.), 2^{nd} Edit., Oxford University Press; 341-361, 2002.
Physicians Desk Reference 1995
Rehman et al., J. Endourol., 17(8):647-657, 2003.
Ringkjob, Acta Neurol. Scand., 44(3):318-322, 1968.
Royal et al., Semin. Nucl. Med., 15:357-376, 1985.
Rubin et al., Cancer Chemother. Rep., 50(5):283-286, 1966.
Satoh et al., Kaku Igaku, 28:429-435, 1991.
Schiff et al., Ann. Neurol., 37(5):583-589, 1995.
Schiff et al., Cancer, 83(8):1593-1601, 1998.
Sendelbeck and Girdis, Drug Metab. Dispos., 13(3):291-295, 1985.
Seo et al., Clin. Imaging, 29(3):172-178, 2005.
Shankar et al., Am. J. Roentgenol., 183(5):1425-1429, 2004.
Shinoda et al., J. Neurooncol., 61:261-265, 2003.
Siperstein and Berber, World J. Surg., 25(6):693-696, 2001.
Slavik, Cancer Treat Rep., 60(6):795-800, 1976.
Teicher et al., Cancer Res., 49(21):5994-5998, 1989.
Wanapirak et al., J. Obstet. Gynaecol. Res., 28(2):71-75, 2002.
Weitzner et al., Cancer, 75(5):1151-1161, 1995.
Winchell et al., J. Nucl. Med., 21:940-946, 1980.
Wintermark et al., Am. J. Neuroradiol., 26(1):104-112, 2005.
Wittink et al., Clin. J. Pain, 20(3):133-142, 2004.
Xu et al., World J. Gastroenterol., 9(12):2686-2689, 2003.
Zhou et al., Am. J. Neuroradiol., 23(1):165-170, 2002.
Zuo et al., World J. Gastroenterol., 10(1):58-61, 2004.

1. A pharmaceutical composition comprising:
a) a C1-C4 alcohol having a partition coefficient of at least 0.1; and
b) a mono- or di-iodinated contrast agent dissolved therein, wherein the contrast agent is soluble in alcohol, DMSO or alcohol/DMSO and has a molecular weight of less than 500.

2. The composition of claim 1, further comprising a chemotherapeutic agent.

3. The composition of claim 2, wherein the C1-C4 alcohol is ethanol.

4. The composition of claim 3, wherein the ethanol is absolute ethanol.

5. The composition of claim 2, wherein the iodinated contrast agent has a molecular weight of less than 400.

6. The composition of claim 2, wherein the iodinated contrast agent is mono iodinated.

7. The composition of claim 2, wherein the iodinated contrast agent is nonionized at physiologic pH.

8. The composition of claim 2, wherein the iodinated contrast agent is selected from the group consisting of iodinated imides, purines and pyrimadines; iodinated benzyls, phenols or benzoic acids; iodinated N-pyridones and iodinated glycerols.

9. The composition of claim 8, wherein the contrast agent is iodinated succinamide, iodinated glycerol, iodouracil or iofetamine.

10. The composition of claim 8, wherein the contrast agent is iodouracil.

11. The composition of claim 2, wherein the chemotherapeutic is a nitrosourea.

12. The composition of claim 11, wherein the nitrosourea is BCNU.

13. The composition of claim 1 or 2, further comprising a solubilizing agent for solubilizing the iodinated contrast agent.

14. The composition of claim 13, wherein the solubilizing agent is DMSO (dimethylsulfoxide).

15. The composition of claim 2, wherein the concentration of chemotherapeutic ranges from 5 mg/ml to 60 mg/ml (w/v).

16. The composition of claim 1 or 2, wherein the iodinated contrast agent comprises 1% to 10% (w/v) of the composition.

17. The composition of claim 1, further defined as absolute ethanol comprising BCNU and iodouracil.

18. The composition of claim 17, further comprising DMSO.

19. The composition of claim 1, 2 or 17, wherein the composition is sterile and contained within a sterile vessel in a metered amount.

20. A method of treating or visualizing a tumor in a subject, comprising intratumorally administering to the tumor a composition in accordance with claim 1.

21. The method of claim 20, wherein the subject is a human.

22. The method of claim 20, wherein the composition is injected into the tumor.

23. The method of claim 20, wherein the tumor is a precancerous tumor, a benign tumor, a cancerous tumor or a metastatic tumor.

24. The method of claim 20, wherein the tumor is present in liver, a lymph node, urinary tract, lung, CNS (brain or spinal cord) or its dural coverings, head and neck, urogenital system, uterus, vertebra, soft tissue, skin, cartilage, bone, or gastrointestinal tract.

25. The method of claim 24, wherein the tumor is a metastatic tumor selected from the group consisting of an extradural metastasis; an intradural metastasis an intramedullary metastasis; and an intravertebral metastasis.

26. The method of claim 24, wherein the tumor is a metastatic tumor originated from a cancer of the breast, lung, blood forming cells, plasma cells, uterus, prostate, or kidney, or an adenocarcinoma, squamous cell carcinoma, renal cell carcinoma, or a myeloma.

27. The method of claim 20, further comprising visualizing the tumor by means of the iodinated contrast agent.

28. The method of claim 20, wherein the volume of the composition administered to the subject is from about 50% to about 100% of the volume of the tumor.

29. A method for preparing a composition in accordance with claim 1, comprising the steps of:
(a) solubilizing the iodinated contrast agent with a solubilizing agent; and
(b) admixing the solubilized iodinated contrast agent with alcohol to form the alcohol contrast agent composition of claim 1.

30. The method of claim 29, wherein the solubilizing agent is DMSO.

31. The method of claim 29, wherein the iodinated contrast agent is iodouracil.

32. The method of claim 29, wherein the composition is formulated to comprise from 5 to 40 mg iodinated contrast agent per milliliter.

33. The method of claim 29, further comprising admixing the alcohol contrast agent composition with a chemotherapeutic.

34. The method of claim 33, wherein the chemotherapeutic is BCNU.

35. A method for treating a spinal metastatic tumor in a subject comprising administering intravertebrally to the tumor an effective amount of a composition comprising a C1-C4 alcohol having a partition coefficient of at least 0.1 and a chemotherapeutic agent dissolved therein.

36. The method of claim 35, wherein the composition further includes a mono- or di-iodinated contrast agent dissolved therein, wherein the contrast agent is soluble in alcohol, DMSO or alcohol/DMSO and has a molecular weight of less than 500.

37. The method of claim 35, wherein the composition is administered intraspinally.

38. The method of claim 35, wherein the spinal metastasis is a spinal axis metastasis.

39. The method of claim 35, wherein the composition is injected into or near the tumor.

40. The method of claim 35, wherein the composition comprises BCNU in ethanol at a concentration of from about 10 mg/ml to about 50 mg/ml (w/v).

41. The method of claim 35, wherein the tumor is an extradural metastasis, an intradural metastasis, an intramedullary metastasis or an intravertebral metastasis.

42. The method of claim 41, wherein the tumor is an extradural metastasis.

43. The method of claim 20 or 35, further comprising administering a second cancer therapy to the subject.

44. The method of claim 43, wherein the second cancer therapy is chemotherapy, focal radiation therapy or a surgery.
